(19) United States
(12) Patent Application Publication
(10) Pub. No.: US 2004/0081621 A1
(43) Pub. Date: Apr. 29, 2004

(54) Optical imaging method, and an apparatus for optical imaging

(76) Inventors: Frank Arndt, Berlin (DE); Arne Hengerer, Erlangen (DE); Thomas Mertelmeier, Erlangen (DE); Marcus Pfister, Erlangen (DE)

Correspondence Address: HARNESS, DICKEY & PIERCE, P.L.C., P.O.BOX 8910, RESTON, VA, 20195, US

(21) Appl. No.: 10/345,960

(22) Filed: Jan. 17, 2003

(30) Foreign Application Priority Data
Jan. 18, 2002 (DE) 10202050.7

(51) Int. Cl. G06K 9/00; A61B 5/00

(52) U.S. Cl. 424/9.6; 382/128; 600/310

(57) ABSTRACT

In an imaging method, an object to be examined is treated with an optically activatable contrast medium and illuminated via a plurality of LEDs. Luminescent light excited by the irradiation is detected by a detector. The LEDs have emission wavelengths preferably different from one another. There are preferably different spectral filter combinations present that respectively include as a structural unit, an excitation filter for selecting an excitation wavelength from the radiation output by the excitation source, and a luminescence filter for filtering out wavelengths above the expected emission maximum of the luminescent light.

The present application hereby claims priority under 35 U.S.C. §119 on German patent application number 10202050.7 filed Jan. 18, 2002, the entire contents of which are hereby incorporated herein by reference.

### FIELD OF THE INVENTION

The invention generally relates to an imaging method, in particular for small animal imaging. Preferably, it relates to a method wherein an object to be examined is treated with an optically activatable contrast medium. The object to be examined, in particular a living one, is then preferably irradiated by an excitation source and luminescent light excited by the irradiation is then detected by a detector.

The invention also generally relates to an apparatus for optical imaging, in particular for small animal imaging and/or for use in an imaging method. The apparatus preferably includes an excitation source for irradiating an object to be examined, in particular a living one, that is treated with an optically activatable contrast medium. It further preferably includes a detector for detecting luminescent light that has been excited by the excitation source.

### BACKGROUND OF THE INVENTION

Optical imaging methods that use contrast media which fluoresce in the near infrared spectral region, in particular, permit examinations of living small animals or of humans. In the case of so-called “small animal imaging”, they are used in addition to methods of magnetic resonance, methods of computer tomography or methods of nuclear medicine for the purpose of biological, medical and pharmaceutical research. They are increasingly being used in pharmaceutical industry as examination methods in the discovery and development of medicaments and active ingredients.

Luminescence-based optical imaging methods are described, for example, in U.S. Pat. No. 5,650,135, EP 0 416 931 A2, U.S. Pat. No. 6,159,445 as well as in a technical article by Umar Mahmood et al., “Near-Infrared Optical Imaging of Protease Activity for Tumor Detection”, Vol. 213, 1999, pages 866-870. With these methods, an optically activatable contrast medium is injected in the object to be examined before the actual imaging phase. Such a contrast medium is composed, for example, of a biological macromolecule, for example an antibody or a peptide, having a high affinity with the target structure to be examined, as well as of a fluorescent dye.

The macromolecule serves in this case as a so-called “metabolic marker”. The effect of this is that the contrast medium, also denoted overall as metabolic marker, either accumulates exclusively in specific regions, for example tumors, inflammations or other specific disease foci; or, although the contrast medium is distributed throughout the body, it can be activated only specially in specific regions, for example by way of specific metabolic functions or enzyme activities. In the latter case, the contrast medium is inert, for example, in healthy tissue and is activated, that is to say converted into a fluorescent state, only in the target tissue to be detected, for example a tumor, by way of disease-correlated metabolic activities.

It is thereby possible substantially to detect functional information about centers thus marked, that is to say the target zone. The observation of the development and temporal variation in such a target zone, for example in conjunction with the administration of a medicament to be tested, permits conclusions to be drawn on the effectiveness and efficiency of the medicament.

Thus, optical fluorescence imaging presupposes a selectively fluorescent contrast medium and thereby differs fundamentally with regard to the physical operating mechanisms from optical imaging methods. These methods utilize the absorption or scattering of the light introduced into the object. Such an absorption-based optical examination method is described, for example, in DE 43 27 798 A1.

In the case of optical fluorescence imaging such as is described, for example, in U.S. Pat. No. 5,650,135, EP 0 416 931 A2 or in the abovementioned technical article by Umar Mahmood et al., the optical excitation of the contrast medium in the object to be examined is performed by an excitation source that emits in the near infrared spectral region, for example. The luminescent or fluorescent radiation returning from the object is detected by an imaging optical detector, for example a photodiode array or a CCD detector. The excitation source and the detector are accommodated for this purpose in a light-proof housing.

A halogen lamp with a downstream bandpass filter is disclosed as excitation source in the technical article by Mahmood et al. Such a halogen lamp disadvantageously has a very high power consumption that generally requires a separate active cooling. The use of halogen lamps is therefore expensive. This also holds for the otherwise customary use of excitation lasers such as is proposed, for example, in U.S. Pat. No. 5,650,135. Lasers and halogen lamps are used because it is thereby possible to generate high light intensities that are required in order to be able to carry out the luminescence examination, which generally exhibits weak signals.

### SUMMARY OF THE INVENTION

It is an object of an embodiment of the invention to specify an imaging method and an imaging apparatus that manage with a low outlay in generating the radiation required to excite the luminescent light, and yet make available a sufficient level of intensity of irradiation for the desired luminescence examination.

In accordance with an embodiment of the invention, the object with respect to the method may be achieved by virtue of the fact that use is made as excitation source of an illumination unit comprising a plurality of LEDs. Here, the abbreviation “LED” stands for “Light-Emitting Diode”, that is to say for a diode-based light-emitting component produced, for example, from semiconductor materials, independently of whether the active medium—as in the case of a conventional gas laser or solid state laser—is incorporated in a resonator, or whether only a conventional light-emitting diode is involved.

An embodiment of the invention proceeds from the consideration that the light power which can be generated by a plurality of LEDs is sufficient for carrying out an optical fluorescence imaging method described at the beginning. Nevertheless, only a comparatively low power assumption need advantageously be assumed in the case of LEDs.

According to a particularly preferred development, LEDs having mutually different emission wavelengths are used. These are, for example, LEDs whose maxima in the emission spectra are different from one another. The half-intensity widths of the emission spectra are preferably greater than 30 nm, in particular greater than 60 nm.

It is preferred to use a plurality of identical LEDs in relation to each emission wavelength used such that the light power can easily be varied for a specific wavelength by switching individual LEDs on or off.

It is particularly advantageous to use LEDs whose emission wavelengths are adapted to the excitation wavelengths desired for a plurality of different contrast media. It is thereby possible in a simple way to change from the examination of one contrast medium to the examination of another contrast medium, without needing to undertake large changes in apparatus. Specifically, by selectively driving the LEDs it is possible to activate only those LEDs that are adapted for a specific contrast medium. The other LEDs are then switched off.

The use of LEDs with emission wavelengths differing from one another is therefore substantially more advantageous than the use of a conventional laser, which emits spectrally in a very narrow fashion only for one or for a few wavelengths that cannot be controlled in a simple way. Specifically, it would be necessary to use either a plurality of lasers or else a very expensive, tunable laser. By contrast, an embodiment of the invention proceeds from the finding that LEDs of high power can be designed at virtually any wavelength of visible light, and sometimes also of infrared light.

According to another advantageous refinement, use is made of LEDs whose emission spectra yield a quasi-continuous emission band in combination.

In order to select an excitation wavelength from the emission band, use is made, in particular, of a filter arrangement that can be introduced into the excitation beam path and whose spectral properties are adapted to the desired contrast medium.

From the spectral point of view, with reference to the generation of light by a halogen lamp such a mode of procedure has—in addition to the advantage of the lower power consumption already mentioned—the further advantage that the required filters need fulfill only modest requirements, because the LEDs used do not emit over a spectral region that is as large as in the case of a halogen lamp. In particular, the suppression need not be so great in the spectral edge regions of the filer. In other words: a spectral preselection can be made by optionally switching on the respective LEDs.

Another preferred refinement of the method provides that light emitted by the LEDs is guided to the object via an assigned optical conductor in each case.

The LEDs are preferably used in an array-like arrangement. They are arranged in the immediate vicinity of one another, in particular, in the array.

Particularly in the case when the light emitted by the LEDs is brought “directly” onto the object to be examined, that is to say without the use of optical filters, optical conductors and/or lenses, it is advantageous that a diffuser is connected downstream of the LEDs in order to achieve a better homogeneous distribution of the wavelengths over the beam emitted by the totality of the LEDs.

With reference to the apparatus mentioned at the beginning, the object with respect to the apparatus is achieved in a first embodiment by virtue of the fact that the excitation source comprises a plurality of LEDs.

The apparatus is preferably used in a method according to an embodiment of the invention. The advantages and refinements mentioned with reference to the method apply analogously to the apparatus.

The LEDs preferably have different emission wavelengths from one another.

In particular, the emission spectra of the LEDs yield a quasi-continuous emission band in combination.

The scope of the invention also further covers an apparatus for optical imaging in a second embodiment. This is based on the finding that the apparatus mentioned at the beginning is particularly advantageous when use is made of a special filter arrangement, specifically if use is made in the imaging method of LEDs whose emission spectra yield a quasi-continuous emission band in combination. As a structural unit, this special filter arrangement has a spectral filter combination as follows:

i) an excitation filter for selecting an excitation wavelength from the radiation output by the excitation source, and

ii) a luminescence filter for filtering out wavelengths above the expected emission maximum of the luminescent light.

Such an apparatus can be operated with particular ease because the two spectral filters are present as a unit provided for a specific contrast medium, being capable, for example, of being introduced into the beam path or removed from the same.

In particular, the excitation filter and the luminescence filter are arranged at or on a common support. A handle can be attached to the support. The supports bear inscriptions, for example, that indicate the contrast medium for which the excitation filter and the luminescence filter are prepared.

In a preferred development, the filter arrangement has a filter wheel for changing one spectral filter combination to another spectral filter combination that is designed in such a way that different spectral filter combinations are used for different angular positions. In the same way as it is possible in the case of a support with a handle for two associated spectral filters to be introduced into or removed from the beam path simultaneously by a single operation, it is possible in the case of the filter wheel for the apparatus to be prepared for a different contrast medium in the case of the filter wheel by only a single driving command, for example triggered by a computer.

The different spectral filter combinations applied at the filter wheel form a set of a plurality of spectral filter combinations which is likewise the subject matter of an embodiment of the invention. The individual spectral filter combinations are prepared for mutually different contrast media, each of the spectral filter combinations comprising an excitation filter for selecting an excitation wavelength, suitable for the respective contrast medium, from the radiation output by an excitation source, as well as a luminescence filter for filtering out wavelengths above the expected emission maximum of the luminescent light emitted by the contrast medium.

The excitation filter and the luminescence filter of one of the spectral filter combinations are preferably respectively arranged at or on a common support. The individual supports are each equipped, for example, with a handle and/or accommodated in a storage box from which they can be removed as required by an operator.

### BRIEF DESCRIPTION OF THE DRAWINGS

Four exemplary embodiments of an apparatus according to the invention are explained in more detail below with the aid of FIGS. 1 to 10. The figures also serve to illustrate the imaging method according to the invention. In the drawings:

FIG. 1 shows an optical imaging apparatus according to the invention in accordance with a first exemplary embodiment,

FIG. 2 shows a cross section through the imaging apparatus of FIG. 1 along the line II/II,

FIG. 3 shows a filter arrangement used with the imaging apparatus of FIG. 1,

FIG. 4 shows a variant relating to the filter arrangement of FIG. 3, using a filter wheel,

FIG. 5 shows a second exemplary embodiment of an imaging apparatus according to the invention,

FIG. 6 shows a third exemplary embodiment of an imaging apparatus according to the invention,

FIG. 7 shows a set of a plurality of spectral filter combinations according to the invention,

FIG. 8 shows a fourth exemplary embodiment of an imaging apparatus according to the invention, only the arrangement of excitation source and detector differing by comparison to FIG. 1 being illustrated,

FIG. 9 shows an alternative design, adapted to the exemplary embodiment of FIG. 8, of a filter arrangement according to the invention, and

FIG. 10 shows an example of an emission spectrum used in the case of an imaging apparatus according to the invention.

### DETAILED DESCRIPTION OF THE PREFERRED EMBODIMENTS

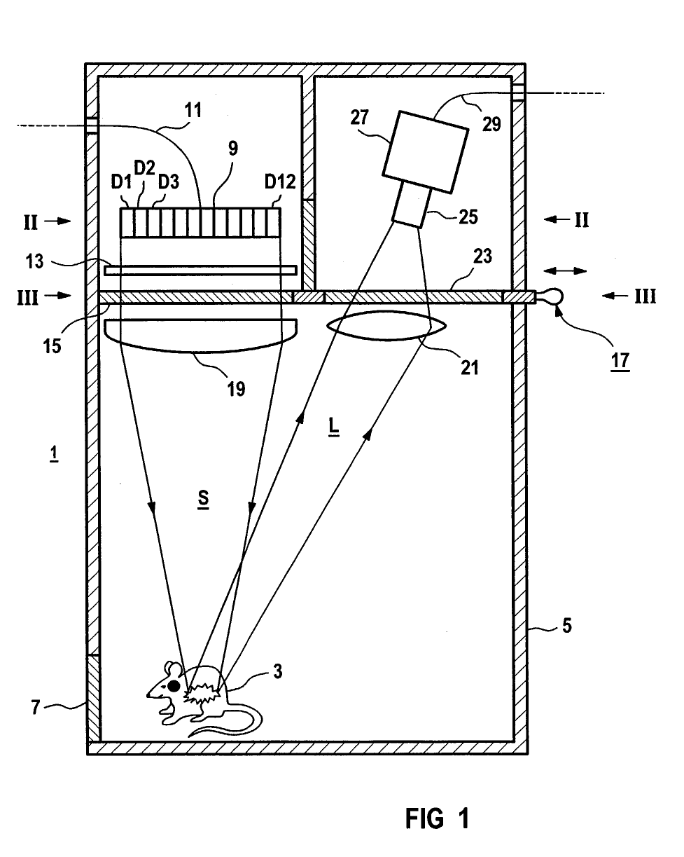

FIG. 1 shows an apparatus 1, suitable for carrying out the imaging method according to an embodiment of the invention, for the optical imaging of an object 3, here a small animal, specifically a mouse. Before the actual imaging step illustrated in FIG. 1, the mouse, which has a breast carcinoma to be visualized, was administered a contrast medium. This is a specific substance, a so-called “metabolic marker”, which either accumulates exclusively in a specific region (for example tumors, inflammations or other specific disease foci), or is distributed throughout the body, but is activated only in special regions, for example by specific enzyme activities.

The fluorescent markers used are very specific, that is to say specific markers interact only with a specific type of tumor. It is also possible to design markers for special applications. The various markers have different optical properties such as excitation and emission wavelengths, and must therefore be handled differently. The following table shows a few of the most commonly used fluorescent dyes with the corresponding excitation and emission wavelengths:

| | Excitation | Emission |
|---|---|---|
| Marker | wavelength | wavelength |
| Indocyanine Green (ICG) | 780 nm | 830 nm |
| CY 5.5 | 675 nm | 694 nm |
| Indocyanine Red (DSRed) | 558 nm | 583 nm |
| Green Fluorescent Protein (GFP) | 489 nm | 508 nm |

In order to carry out the actual imaging method step, the object 3 thus treated with a marker is brought via a flap 7 into a light-proof housing 5. Arranged in the left-hand upper partial chamber of this housing 5 as excitation source 9 is an array of LEDs or light-emitting diodes D1, D2, . . . , D12 that is supplied by a power pack (not shown) via an electric line 11. Connected downstream in the direction of propagation of the radiation S emitted by the diodes D1, D2, . . . , D12 is a diffuser 13 that serves a purpose of spatially mixing the different wavelengths emitted by the diodes D1, D2, . . . , D12. Immediately thereafter, the radiation S strikes an excitation filter 15 for selecting an excitation wavelength from the radiation S output by the excitation source 9. The excitation filter 15 is part of a filter arrangement 17 that also plays a role on the detection side, as will be explained further below.

The radiation S passing the excitation filter 15 is projected by a condenser 19 onto the desired examination region of the mouse. Luminescent light L excited in the object 3 by the irradiation passes to a lens 21 of the apparatus 1 that is arranged laterally next to the condenser 19. Subsequently, the luminescent light L passes a luminescence filter 23 that is situated in a structural unit with the excitation filter 15 and forms the filter arrangement 17 together therewith. The luminescence filter 23 serves to filter out or suppress wavelengths above the expected emission maximum in the luminescent light L.

The apparatus 1 also comprises a detector 27 for detecting the luminescent light L with the aid of an upstream lens 25. The electric signals generated by the detector 27 are fed via a line 29 to an image processing system (not illustrated separately) on whose display screen an image of the examination region of the mouse 3 is visualized, the regions marked by the selective contrast medium, in particular occupied by carcinomas, being visualized effectively. The detector 27 is arranged in the right-hand upper chamber of the housing 5 that is separated from the excitation source 9 by a wall screening off scattered light.

The emission wavelengths of the LEDs D1, D2, . . . , D12 of the excitation source 9 are different from one another.

In accordance with an exemplary embodiment that is not illustrated, the excitation source 9 comprises, for example, a total of 9 LEDs that are arranged in accordance with a 3×3-matrix. The first column comprises three LEDs emitting at 600 nm, the middle column comprises three LEDs emitting at 650 nm, and the right-hand column comprises three LEDs emitting at 675 nm.

The optical output power of the LEDs is in the region of 5-10 mW. It is also possible to use LEDs that emit in the NIR with a power of up to 1 W.

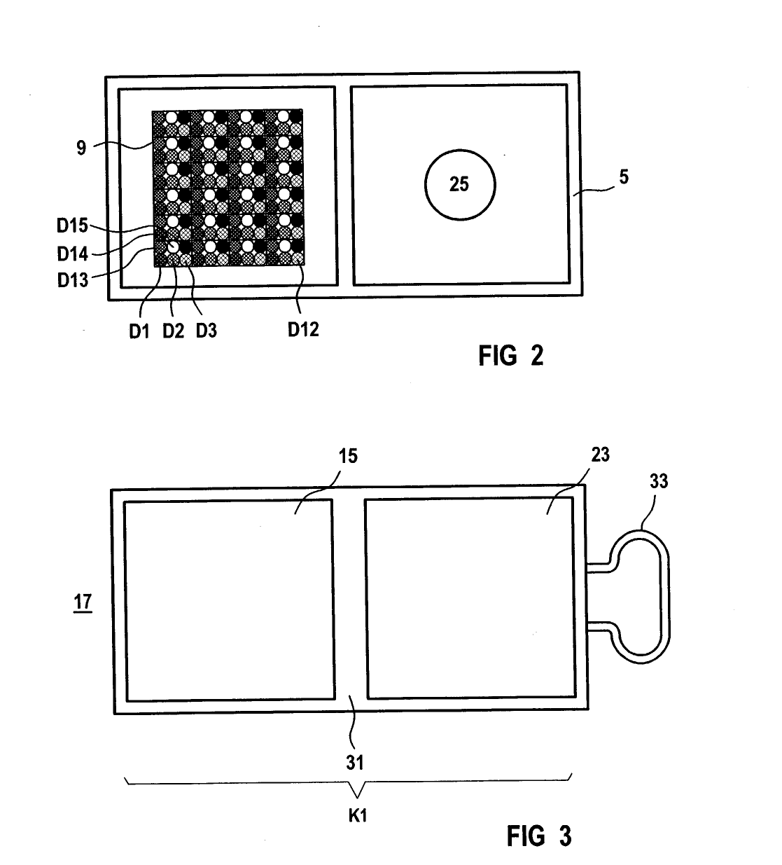

An alternative refinement of the excitation source 9 may be seen from the cross-sectional illustration of FIG. 2. A plurality of diode groups are arranged in array-like or matrix-like fashion in this exemplary embodiment. Each row and each column comprises a plurality of diode groups that preferably resemble one another. Each diode group comprises a plurality of preferably respectively identical light-emitting diodes with mutually different emission wavelengths. In the example illustrated, the excitation source 9 has 6×4, that is to say a total of 24, diode groups. Each of the four columns comprises six diode groups which, for their part, respectively have in turn six different LEDs. The arrangement of the LEDs D1, D2, . . . , D12 is unbroken. One of the diode groups—comprising six diodes D1, D2, D3, D13, D14, D1S emitting at respectively different emission wavelengths—is indicated by way of example.

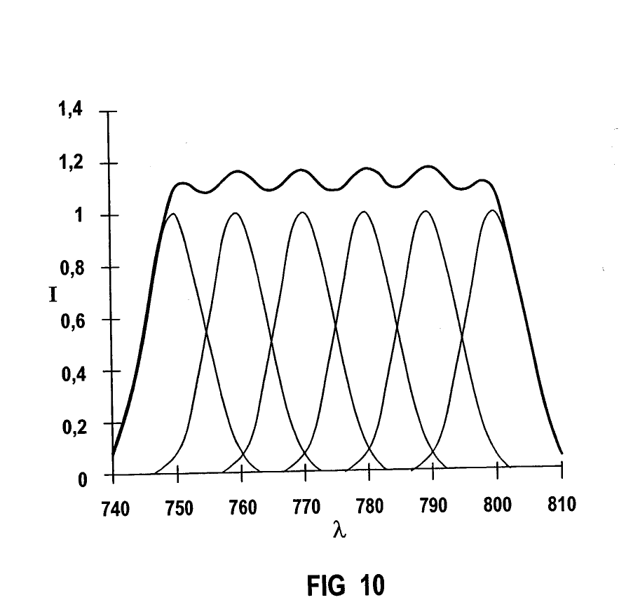

The LEDs of high power have a spectral half-intensity width of approximately 40 nm. Since they have different wavelength maxima and can be operated simultaneously in the immediate vicinity of one another, it is possible to add the Gaussian distributions of their individual emission spectra to form a total spectrum. This is illustrated in FIG. 10 in which the profiles of the intensity I of the individual LEDs are plotted against the wavelength λ. The starting point here is six Gaussian distributions, shifted by 10 nm in each case, with half-intensity widths of 40 nm, it thereby being possible to generate a virtually continuous spectrum in the range of 745 nm to 805 nm. In the case of the use of LEDs with larger half-intensity widths, a smaller number of LEDs is required, or a yet more continuous or wider spectrum can be produced with no change in the number.

The filter arrangement 17 of the apparatus 1 is illustrated in more detail in FIG. 3 in the dismantled state. The filter arrangement 17 has a support 31 with a handle 33. Both the excitation filter 17 and the luminescence filter 23 are arranged on the support 31, specifically next to one another. The support 31 can be plugged into an opening of the housing 5 by means of the handle 33. The support 31 bears a description or an indication of a specific marker or the optical properties thereof onto which marker or properties the filter arrangement 17 is tuned. By plugging in different filter arrangements 17, it is therefore possible to change easily from an examination with one contrast medium to an examination with another contrast medium. In this arrangement, each of the filter arrangements 17 can be understood as a two-part plug-in filter whose first part allows the desired excitation wavelength to pass via the excitation filter 15, and whose other part allows an expected emission wavelength to pass via the luminescence filter 23. The filters 15, 23 are designed in each case as interference filters.

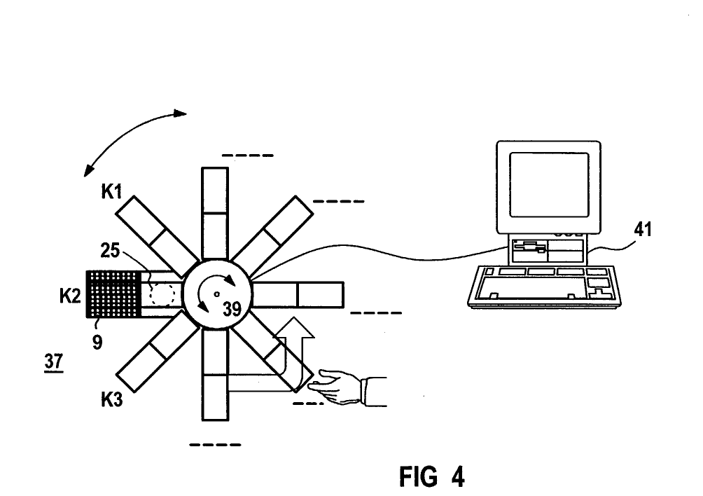

The exemplary embodiment illustrated in FIG. 4 can be understood such that a plurality of filter arrangements 17 with mutually different excitation filters 15 and luminescence filters 23 are designed as a filter wheel 37. A plurality of spectral filter combinations K1 (see also FIG. 3), K2, K3 are arranged in the shape of a star on a rotary member 39 in such a way that different spectral filter combinations K1, K2, K3 are used for different angular positions of the rotary member 39. Each of the spectral filter combinations K1, K2, K3 comprises as a structural unit another combination in each case of an excitation filter 15 and a luminescence filter 23. The filter wheel 37 can engage in a free space, accessible from outside the housing 5, in such a way that the excitation filter 15 and luminescence filter 23 can be positioned as illustrated in FIG. 1.

The filter wheel 37 can be driven by a computer 41 as regards a rotary movement. The exchange of the spectral filter combinations K1, K2, K3, that is to say the adaptation of the system to different markers, is performed simply by rotating the filter wheel 37. This can also be performed manually. It is advantageous when using the illustrative computer 41 that in the case when animal experiments or a protocol of the experiments with a specific animal are stored in a database, the computer 41 uses the database information of an experiment or animal that includes the marker used to select the respectively required spectral filter combination K1, K2, K3 directly and, in particular, without special intervention by the user.

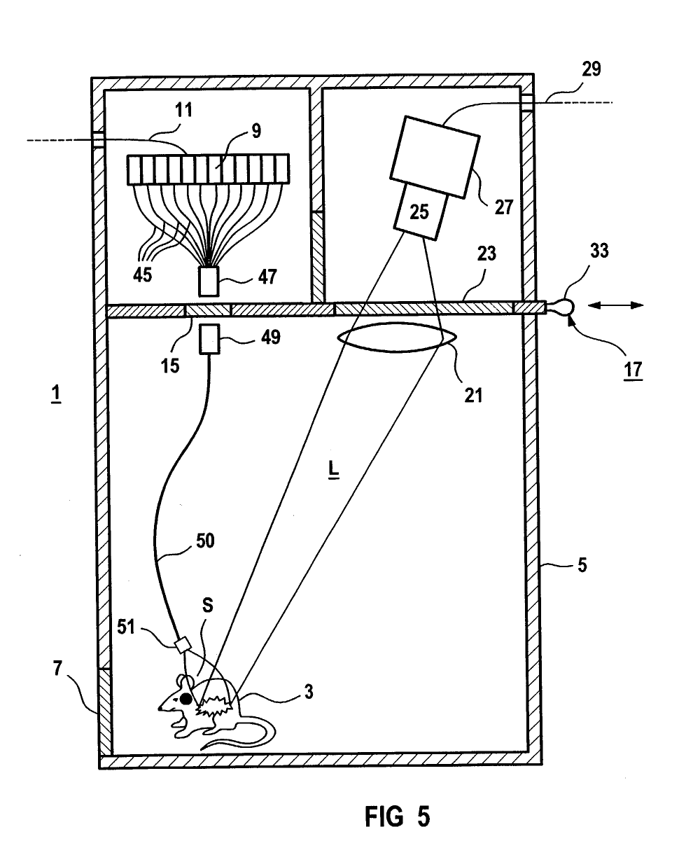

In the exemplary embodiment illustrated in FIG. 5 of an imaging apparatus 1 according to an embodiment of the invention, the radiation S is brought to the object 3 by optical waveguides, in contrast to FIG. 1. For this purpose, each diode D1, D2, . . . , D12 is assigned a separate optical conductor 45 that picks up the light emanating from the respective diode D1, D2, . . . , D12 and leads it to a filter or a first coupler 47. Collected in such a way, the light passes the excitation filter 15 and is led by a downstream second coupler 49 and an optical fiber 50 to a lens 51 near the object. From there, the light or the radiation S passes to the object 3. Otherwise, the exemplary embodiment of FIG. 5 is identical to that in accordance with FIG. 1. The LED array can also be placed outside the light-proof housing 5 in the case of the exemplary embodiment illustrated in FIG. 5.

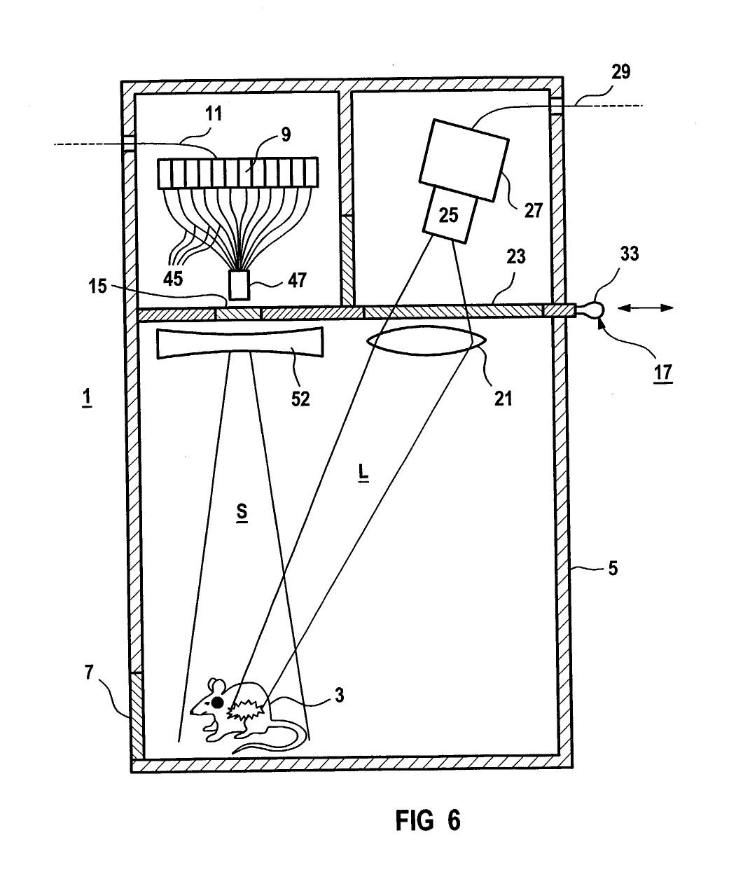

As in the case of the abovementioned exemplary embodiment, it is also possible in the case of the exemplary embodiment in accordance with FIG. 5 (and therefore of the following FIG. 6) to turn on or off individual LEDs, of which a plurality are present in relation to each emission wavelength used. This is done in order to adapt the power of the overall array, that is to say the excitation source 9, to the fluorescent dye and/or to the organism to be examined.

The third exemplary embodiment, illustrated in FIG. 6, of an imaging device according to an embodiment of the invention is largely identical to the exemplary embodiment illustrated in FIG. 5, the difference from this being that the light collected by the first coupler 47 is projected onto the object 3 not by an optical fiber, but via a downstream expanding lens 52—this being done in a way similar to FIG. 1.

The exemplary embodiments of FIGS. 5 and 6 have the advantage that the LEDs can also be fitted outside the housing 5 and can thus easily be exchanged without having to open the housing 5 of the apparatus 1.

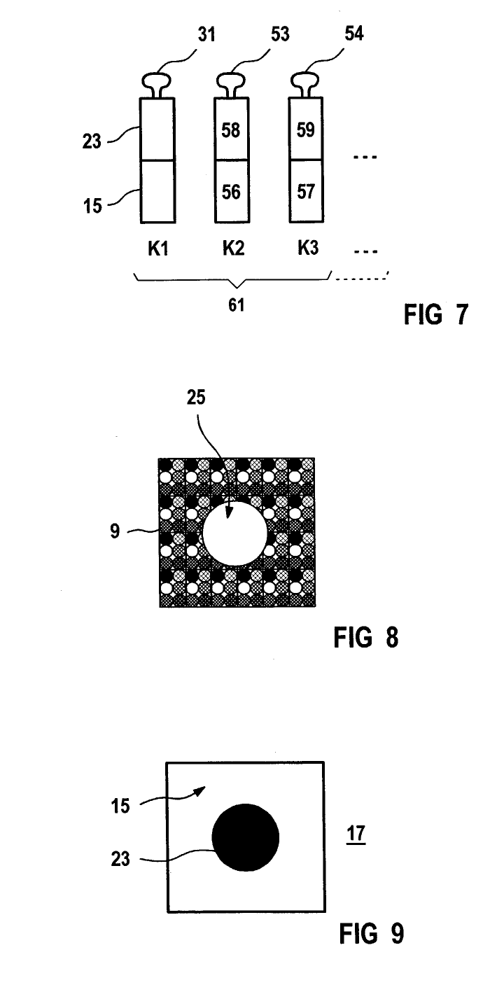

Different spectral filter combinations K1, K2, K3 can be pushed or plugged into the apparatuses 1 in accordance with FIGS. 1, 5 and 6. A set 61 comprising three or more such spectral filter combinations K1, K2, K3 is illustrated in FIG. 7. Each of the spectral filter combinations K1, K2, K3 is implemented by a support 31, 53, 54 on which an excitation filter 15, 56, 57 is respectively arranged and a luminescence filter 23, 58, 59 is respectively arranged. The supports 31, 53, 54 are each of identical construction and can be distinguished from one another as a rule only be different inscriptions and/or colorings, and can be selected for an examination with the aid of a specific desired marker or contrast medium.

An alternative to the relative arrangement, as illustrated in FIG. 2, of the excitation source 9 and the detector 27 with respect to one another is the exemplary embodiment, illustrated in FIG. 8, of an imaging apparatus 1 according to an embodiment of the invention, which is illustrated only with regard to this detail. In this alternative, the CCD camera lens 25 is integrated into the LED array of the excitation source 9.

A filter arrangement 17 prepared for this alternative is illustrated in FIG. 9. It is likewise designed as a plug-in filter, although here the division is performed into an inner region for the luminescence filter 23 (emission wavelength) and into a surrounding outer region for the excitation filter 15 (excitation wavelength).

The idea on which the invention is based proceeds by using a wide continuous illumination spectrum in optical imaging based on luminescence in order to allow nothing but the absolutely necessary excitation wavelength to pass by filtering from this continuous spectrum.

The imaging apparatuses and the imaging method according to an embodiment of the invention permit an exceptionally rapid execution of batteries of tests and experiments, particularly in the pharmaceutical industry during development of medicaments. The apparatuses according to the invention are flexible and are suitable, without major technical changes between the individual experiments, for exciting common markers and detecting their emission wavelengths. Moreover, it is possible to work with newly developed markers that have hitherto unusual optical properties without large technical and expensive changes.

It is possible in the case of illumination with the aid of LEDs of different wavelengths to undertake the selection of a specific excitation wavelength from the continuous illumination spectrum produced simply by inserting a filter of the desired excitation wavelength upstream of the light source. The same holds for the selection of an emission wavelength by inserting a filter upstream of the camera. Imaging experiments with different markers can therefore easily be prepared by exchanging the filters. Instead of a new electrooptic illumination unit, only a new filter need be produced for new markers.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

What is claimed is:

1. An imaging method, comprising:
treating an object to be examined with an optically activatable contrast medium;
irradiating the object to be examined by an excitation source; and
detecting luminescent light excited by the irradiation by a detector, wherein an illumination unit including a plurality of LEDs is used as excitation source.

2. The imaging method as claimed in claim 1, wherein the LEDs include mutually different emission wavelengths.

3. The imaging method as claimed in claim 2, wherein a plurality of identical LEDs are used in relation to each emission wavelength used.

4. The imaging method as claimed in claim 2, wherein the emission wavelengths of the LEDs are adapted to the excitation wavelengths desired for a plurality of different contrast media.

5. The imaging method as claimed in claim 2, wherein the emission spectra of the LEDs yield a quasi-continuous emission band in combination.

6. The imaging method as claimed in claim 5, wherein a filter arrangement is used in order to select an excitation wavelength from the emission band, the filter arrangement being introducable into the excitation beam path and including spectral properties adapted to the contrast medium.

7. The imaging method as claimed in claim 1, wherein light emitted by the LEDs is guided to the object via an assigned optical conductor.

8. The imaging method as claimed in claim 1, wherein the LEDs are used in an array-like arrangement.

9. The imaging method as claimed in claim 1, wherein a diffuser is connected downstream of the LEDs.

10. An apparatus for optical imaging, comprising:
an excitation source for irradiating an object to be examined, wherein the object is treated with an optically activatable contrast medium;
a detector for detecting luminescent light that has been excited by the excitation source, wherein the excitation source includes a plurality of LEDs.

11. The apparatus as claimed in claim 10, wherein the LEDs include mutually different emission wavelengths.

12. The apparatus as claimed in claim 10, wherein the emission spectra of the LEDs yield a quasi-continuous emission band in combination.

13. An apparatus for optical imaging, comprising:
an excitation source for irradiating an object to be examined, wherein the object is treated with an optically activatable contrast medium;
a detector for detecting luminescent light that has been excited by the excitation source; and
a filter arrangement including, as a structural unit, a spectral filter combination as follows,
an excitation filter for selecting an excitation wavelength from radiation output by the excitation source, and
a luminescence filter for filtering out wavelengths above the expected emission maximum of the luminescent light.

14. The apparatus as claimed in claim 13, wherein the excitation filter and the luminescence filter are arranged at least one of at and on a common support.

15. The apparatus as claimed in claim 13, wherein the filter arrangement includes a filter wheel for changing one spectral filter combination to another spectral filter combination that is designed in such a way that different spectral filter combinations are used for different angular positions.

16. A set of a plurality of spectral filter combinations that are prepared for mutually different contrast media, each of the spectral filter combinations comprising:
an excitation filter for selecting an excitation wavelength, suitable for the respective contrast medium, from irradiation output by an excitation source; and
a luminescence filter for filtering out wavelengths above the expected emission maximum of the luminescent light emitted by the contrast medium.

17. The set as claimed in claim 16, wherein the excitation filter and the luminescence filter of one of the spectral filter combinations are respectively arranged at least one of at and on a common support.

18. An imaging method as claimed in claim 1, wherein the imaging method is for small animal imaging.

19. The imaging method as claimed in claim 3, wherein emission wavelengths of the LEDs are adapted to the excitation wavelengths desired for a plurality of different contrast media.

20. The imaging method as claimed in claim 3, wherein the emission spectra of the LEDs yield a quasi-continuous emission band in combination.

21. The imaging method as claimed in claim 4, wherein the emission spectra of the LEDs yield a quasi-continuous emission band in combination.

22. The imaging method as claimed in claim 2, wherein light emitted by the LEDs is guided to the object via an assigned optical conductor.

23. The imaging method as claimed in claim 2, wherein the LEDs are used in an array-like arrangement.

24. The imaging method as claimed in claim 2, wherein a diffuser is connected downstream of the LEDs.

25. An apparatus for performing the imaging method of claim 1.

26. The apparatus as claimed in claim 10, wherein the apparatus is for small animal imaging.

27. The apparatus as claimed in claim 11, wherein the emission spectra of the LEDs yield a quasi-continuous emission band in combination.

28. The apparatus as claimed in claim 13, wherein the apparatus is for small animal imaging.

29. The apparatus as claimed in claim 14, wherein the filter arrangement includes a filter wheel for changing one spectral filter combination to another spectral filter combination that is designed in such a way that different spectral filter combinations are used for different angular positions.

30. An imaging apparatus, comprising:
means for treating an object to be examined with an optically activatable contrast medium;
means for irradiating the object to be examined by an excitation source; and
means for detecting luminescent light excited by the irradiation by a detector, wherein an illumination unit including a plurality of LEDs is used as excitation source.

31. An apparatus for optical imaging, comprising:
excitation means for irradiating an object to be examined, wherein the object is treated with an optically activatable contrast medium;
detection means for detecting luminescent light that has been excited by the excitation means, wherein the excitation means includes a plurality of LEDs.

32. An apparatus for optical imaging, comprising:
excitation means for irradiating an object to be examined, wherein the object is treated with an optically activatable contrast medium;
detection means for detecting luminescent light that has been excited by the excitation means; and
a filter arrangement including, as a structural unit, a spectral filter combination as follows,
excitation filtering means for selecting an excitation wavelength from radiation output by the excitation means, and
luminescence filter means for filtering out wavelengths above the expected emission maximum of the luminescent light.

33. A set of a plurality of spectral filter combinations that are prepared for mutually different contrast media, each of the spectral filter combinations comprising:
excitation filter means for selecting an excitation wavelength, suitable for the respective contrast medium, from irradiation output by an excitation source; and
luminescence filter means for filtering out wavelengths above the expected emission maximum of the luminescent light emitted by the contrast medium.
